(19) United States
(12) Patent Application Publication
(10) Pub. No.: US 2024/0420368 A1
(43) Pub. Date: Dec. 19, 2024

(54) TREATMENT APPARATUS AND METHOD FOR DETERMINING AN EYE ORIENTATION

(71) Applicant: SCHWIND EYE-TECH-SOLUTIONS GMBH, Kleinostheim (DE)

(72) Inventors: Samuel ARBA MOSQUERA, Aschaffenburg (DE); Nico TRIEFENBACH, Mainaschaff (DE); Thomas KLINNER, Glattbach (DE)

(21) Appl. No.: 18/740,852

(22) Filed: Jun. 12, 2024

(30) Foreign Application Priority Data
Jun. 15, 2023 (DE) 10 2023 115 691.8

(51) Int. Cl. G06T 7/73; A61F 9/008

(52) U.S. Cl. CPC G06T 7/73; A61F 9/008; A61F 2009/00846; G06T 2207/30041

(57) ABSTRACT

The invention relates to a treatment apparatus (10) and to a method for determining an eye orientation of an eye (16). The treatment apparatus (10) includes at least one ophthalmological laser (12) for irradiating the eye (16) by laser radiation (20); a capturing device (24) with at least one camera (26), wherein the capturing device (24) is formed to capture eye recording data by the at least one camera (26); and a computing device (18), which is formed to ascertain an eye orientation from the eye recording data, wherein the eye orientation includes a distance (3D), a vertical displacement (1D) and a horizontal displacement (2D) of the eye (16) in relation to a preset reference point of the ophthalmological laser (12) as well as a cyclotorsion (6D), a vertical rotation (4D) and a horizontal rotation (5D) of the eye (16), wherein the computing device (18) is further formed to generate control data for controlling the treatment apparatus (10) depending on the ascertained eye orientation.

### FIELD

The invention relates to a treatment apparatus for treating a human and/or animal eye. Furthermore, the invention relates to a method for determining an eye orientation of a human or animal eye, to a computer program comprising commands, which cause the treatment apparatus to execute the method, as well as to a computer-readable medium, on which the computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device can for example be formed such that laser pulses effect a photodisruption and/or ablation in a focus situated within the organic tissue to remove a tissue, in particular a tissue lenticule, from the cornea.

Due to displacements of the eye to a reference point of the laser, however, it can occur that undesired effects, in particular decentrations and aberrations, in particular chromatic aberrations, are generated in a treatment. In order to center an eye for the treatment, eye tracking systems (“eye trackers”) or pupil tracking systems (“pupil trackers”) are known, which can recognize translational displacements of the eye to provide a compensation for the displacement. However, conventional eye tracking systems do not capture all of the possible causes of the decentration of the eye, wherefore undesired treatment effects can further occur despite of a compensation for an assumed translational displacement.

### SUMMARY

Therefore, it is the object of the invention to improve the capture of an eye orientation of an eye for the treatment with an ophthalmological laser.

This object is solved by the invention described herein. Advantageous embodiments of the invention are disclosed in the following description, figures, and claims.

The invention is based on the idea that the eye orientation is ascertained in six dimensions, wherein a rotation of the eye in x-, y- and z-direction is also determined besides the translational displacements in x-, y- and z-direction to be able to determine the cause of a decentration in improved manner. Herein, the distinction if the eye is displaced due to a translation or a rotation, can in particular significantly contribute to a compensation for the resulting effects.

An aspect of the invention relates to a treatment apparatus for treating a human and/or animal eye. The treatment apparatus includes at least one ophthalmological laser for irradiating the eye by laser radiation, a capturing device with at least one camera, wherein the capturing device is configured to capture eye recording data by the at least one camera, and a computing device, which is configured to ascertain an eye orientation from the eye recording data, wherein the eye orientation includes a distance, a vertical displacement and a horizontal displacement of the eye in relation to a preset reference point of the ophthalmological laser as well as a cyclotorsion, a vertical rotation and a horizontal rotation of the eye. Furthermore, the computing device is configured to generate control data for controlling the treatment apparatus depending on the ascertained eye orientation.

In other words, the treatment apparatus, which is configured for treating a human or animal eye, can include at least one ophthalmological laser, in particular a photoablative and/or photodisruptive laser and/or a laser for generating a laser-induced refractive index change and/or a laser for increasing a crosslinking of corneal tissue.

Furthermore, the treatment apparatus can comprise at least one camera, in particular multiple cameras, which can be provided in a capturing device of the treatment apparatus, wherein the camera is configured to capture eye recording data of the eye. This means that the eye recording data can include one or more recordings of the eye, which can be located in a treatment area at the treatment apparatus. The eye recording data can be recorded in an optical or visible spectral range and/or an infrared spectral range.

A computing device of the treatment apparatus, which can in particular be formed as a computer comprising one or more processors, in particular microprocessors, can then evaluate the eye recording data and determine the eye orientation. Herein, the computing device can not only ascertain a translational displacement in x-direction (horizontal displacement), y-direction (vertical displacement) and z-direction (distance) of the orientation of the eye, but can also ascertain rotations of the eye. On the one hand, a cyclotorsion, that is a rotation around a z-axis, which can in particular extend through the reference point of the laser, can be ascertained as the rotational displacement, as well as tilts or rotations of the eye along an arc in x-direction (vertical rotation) and y-direction (horizontal rotation).

In order to determine the rotational movement, multiple strategies can be applied for evaluating the eye recording data. On the one hand, landmarks or preset structures can be recognized in the eye, wherein it can be differentiated due to a changed representation of these landmarks or preset structures in the captured eye recording data if a displacement or decentration due to a rotation or a translation of the eye is present. For determining the landmarks, a recording of the eye, in particular a diagnostic image of the eye, with known eye orientation can for example be used, wherein the landmarks of the diagnostic image can be compared to the eye recording data of the capturing device to determine the change. Herein, circular structures have proven to be particularly suitable landmarks, such as for example, a pupil edge or a limbus (transition from iris to sclera) of the eye. For example, if these circular structures are represented elliptically or ovally deformed in the eye recording data, a rotation can be present. However, if the structure maintains the circular shape, but is outside of the center of the reference point, thus, only a translational displacement can be present. Herein, the reference point may be a point in or on the eye, for example a corneal vertex and/or a pupil central point. Alternatively or additionally, the reference point can also be a neutral position or zero position of the laser.

As a further strategy for determining the eye orientation, the capturing device can comprise multiple cameras, which can record the eye from different directions. This means that the cameras can be oriented to the eye at different known angles. Based on the eye recording data, which has been recorded from different directions, a rotation and/or translation of the eye can then be inferred.

As a further strategy for determining the eye orientation, it can also be provided that the treatment apparatus, in particular the capturing device, includes a projection unit, which can radiate a light signal with a preset projection pattern to the eye, wherein a reflection of the projection pattern can be captured by the camera. Depending on a deformation of the recorded reflection of the projection pattern, a translation and/or rotation of the eye can then be calculated.

The ascertained eye orientation can then be used to generate control data for controlling the treatment apparatus or to adapt predetermined control data, in particular such that originally planned irradiation positions are adapted to the ascertained eye orientation. The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

By the invention, the advantage arises that better treatment results can be achieved.

The invention also includes embodiments, by which additional advantages arise.

In an embodiment, the capturing device includes a projection unit, which is configured to radiate a projection pattern, wherein the computing device is configured to ascertain the eye orientation depending on the projection pattern captured in the eye recording data. For example, it can be provided that the projection pattern is a stripe pattern or grid pattern with preset distances between the stripes. In other words, a previously known projection pattern can be radiated to the eye by a projection unit (projector), wherein a reflection of the projection pattern, which is reflected from the eye, can be detected by the capturing device. If a stripe pattern is used, a distance to the projection unit and thus of the eye to the reference point can be determined due to distances between the stripes. Furthermore, a rotation in horizontal and/or vertical direction can be ascertained by a progression of the distances. For example, if the distance becomes larger on one side of the projection pattern than on the other side in the reflection of the stripes, a rotation can be present. In particular, this can be used together with a captured pupil position or a captured center of the limbus to thus determine a portion of the translation and a portion of the rotation. By this embodiment, the advantage arises that a rotation of the eye can be determined in improved manner.

In a further embodiment the capturing device comprises at least two cameras, which are arranged to record the eye recording data from different angles. This means that the capturing device comprises at least two cameras, in particular more than two cameras, which are configured and arranged to record the eye from multiple directions or angles. From the volume information thus obtained, a translation and/or rotation of the eye to the reference point can then be ascertained. For example, if the eye is decentered in the eye recording of one camera and centered in the eye recording of the other camera, a rotation into the corresponding direction of the camera can be inferred. By this embodiment, the advantage arises that an eye orientation can be determined in improved manner, which improves the provision of the control data.

Alternatively or additionally, it is provided that the camera is configured as a plenoptic camera. The plenoptic camera, which is also referred to as light field camera, can capture a direction of the incident light beams, from which information about the image depth can be determined, besides the usual 2 image dimensions. Accordingly, the eye orientation can be determined from the eye recording data, which has been recorded by the plenoptic camera.

In a further embodiment, landmarks of the eye are predetermined, wherein the computing device is configured for determining the eye orientation depending on a position of the landmarks in the eye recording data. For example, a pupil central point can serve as the landmark on the one hand, by which a displacement in horizontal and/or vertical direction can be ascertained. Furthermore, a structure of the iris can for example be predetermined, from which a cyclotorsion can be ascertained. Furthermore, circular structures in the eye, in particular a limbus, can for example be used as the landmark to ascertain a rotation and/or a distance of the eye. Thus, a predetermined diameter of the limbus of the eye can for example be compared to a diameter of the limbus in the eye recording data to determine the distance of the eye to the treatment apparatus, wherein the diameter or the radius increases with decreasing distance. For determining the rotation in vertical and/or horizontal direction, it can be examined if the circular structure, in particular the limbus, has an elliptical shape in the eye recording data, wherein the rotational direction and/or a rotational angle can be provided depending on the shape of the arising ellipse. Thus, by a pupil position or a position of a limbus center in combination with the deformation of the limbus and/or the pupil in the eye recording data, it can in particular be inferred how high a translational portion and how high a rotational portion of the eye are. By this embodiment, the advantage arises that the eye orientation can be ascertained with predetermined structures or landmarks of the eye and only one camera.

In a further embodiment, the computing device is configured to monitor a centering of the eye on a contact element of the treatment apparatus with the control data. This means that an approaching procedure of the eye to a contact element can be monitored, wherein the control data can serve to output signals if the eye is decentered by translation and/or rotation. In particular, the control data can be used to start a fixing device for automatically fixing the eye if the centering corresponds to a preset centering condition both in translational direction and in rotational direction.

In a further embodiment, the computing device is configured to adapt irradiation positions of the laser beam of the ophthalmological laser with the control data, in particular during a treatment. This means that the originally planned irradiation positions can be adapted based on a current eye orientation to thus irradiate the planned positions also in case of a decentration of the eye. Hereby, a treatment can be improved.

A further aspect of the invention relates to a method for determining an eye orientation of a human and/or animal eye for a treatment apparatus with an ophthalmological laser, wherein eye recording data of the eye is captured by a capturing device, which includes at least one camera, wherein an eye orientation is ascertained from the eye recordings by a computing device, wherein a distance, a vertical displacement and a horizontal displacement of the eye in relation to a preset reference point of the ophthalmological laser as well as a cyclotorsion, a vertical rotation and a horizontal rotation of the eye are determined as the eye orientation, wherein control data for controlling the treatment apparatus is generated by the computing device depending on the ascertained eye orientation. Herein, the same advantages and possibilities of variation as in the treatment apparatus result.

The method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect relates to a computing device or a control device, which is configured to perform the steps of at least one embodiment of one or both of the previously described methods. Thereto, the computing device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the method are encoded. The program code can include the control data for the respective laser. The program code can be executed by the computing unit, whereby the computing device is caused to execute the respective embodiment. The computing device can be configured as a control chip or control unit. The computing device can for example be encompassed by a computer or computer cluster.

A further aspect of the invention relates to a computer program. The computer program includes commands, which for example form a program code. The program code can include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. The storage medium is for example formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a treatment apparatus according to an exemplary embodiment.

FIG. 2 depicts a treatment apparatus according to a further exemplary embodiment.

FIG. 3 depicts a treatment apparatus according to a further exemplary embodiment.

FIG. 4 depicts a representation of eye orientations of an eye.

In the figures, identical or functionally identical elements are provided with the same reference characters.

### DETAILED DESCRIPTION

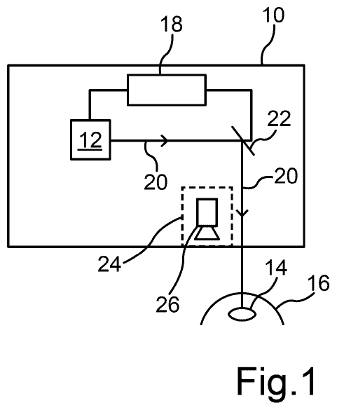

FIG. 1 shows a schematic representation of a treatment apparatus 10 with an ophthalmological laser 12 for removing a tissue 14 from a human or animal eye 16, in particular a cornea, by photodisruption and/or ablation. For example, the tissue 14 can represent a lenticule or also volume body, which can be separated from the cornea of the eye 16 for correcting a visual disorder by the laser 12. A correction profile or a geometry of the tissue 14 to be removed can be provided by a computing device 18 or control device, in particular in the form of control data, such that the laser 12 emits pulsed laser pulses in a pattern predefined by the control data into the cornea of the eye 16, to remove the tissue 14. Alternatively, the computing device 18 can be a computing device 18 external with respect to the treatment apparatus 10.

Furthermore, FIG. 1 shows that the laser beam 20 generated by the laser 12 can be deflected towards the eye 16 by a beam deflection device 22 such as for example a rotation scanner, to remove the tissue 14. The beam deflection device 22 can also be controlled by the computing device 18 to remove the tissue 14.

The illustrated laser 12 may be a photodisruptive and/or photoablative laser, which is formed to emit laser pulses in a wavelength range between 300 nanometers and 1400 nanometers, for example between 700 nanometers and 1200 nanometers, at a respective pulse duration between 1 femtosecond and 1 nanosecond, for example between 10 femtoseconds and 10 picoseconds, and a repetition frequency of greater than 10 kilohertz, for example between 100 kilohertz and 100 megahertz. In addition, the computing device 18 optionally comprises a storage device (not illustrated) for at least temporary storage of at least one control dataset, wherein the control dataset or datasets include(s) control data for positioning and/or for focusing individual laser pulses in the cornea.

In order that predetermined treatment positions in the eye 16 can be addressed by the laser beam 20, an eye orientation of the eye 16 in a reference point of the laser has to coincide with a predetermined eye orientation or the eye orientation in relation to the reference point has to be known or determinable to direct the laser beam 20 to the corresponding treatment positions by the beam deflection device 22. In order to determine an eye orientation of the eye 16, the treatment apparatus 10 can comprise a capturing device 24. Herein, it can be provided that the capturing device 24 includes at least one camera 26, which records recordings of the eye 16 in a visible and/or infrared spectral range and provides them to the computing device 18 as eye recording data.

Further, the computing device 18 can be formed to evaluate the eye recording data and thus to provide a distance, a vertical displacement and a horizontal displacement of the eye 16 in relation to the reference point, which can be preset by a neutral position of the beam deflection device 22, as the eye orientation. Furthermore, the computing device 18 can be formed to determine a cyclotorsion, this means a rotation of the eye 16 around a preset axis of the laser beam 20 in neutral position of the beam deflection device 22, as well as a vertical rotation and a horizontal rotation of the eye 16 by the eye recording data.

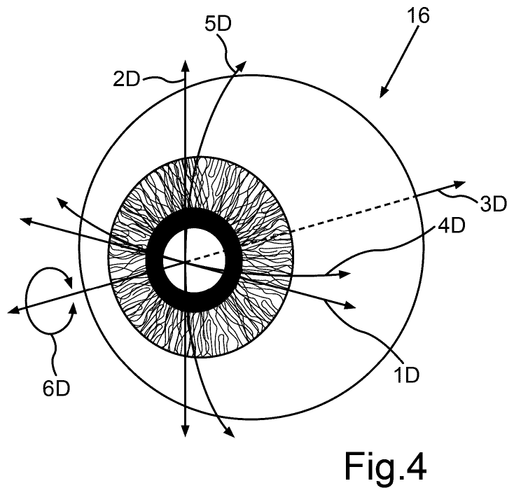

The eye orientation for the eye 16 with the associated directions is for example illustrated in FIG. 4. Herein, a first dimension is provided by a horizontal displacement 1D, a second dimension by a vertical displacement 2D and a third dimension by a distance or a lateral displacement 3D, which together provide a translational displacement of the eye 16 with respect to the treatment apparatus 10 or the reference point. Furthermore, a horizontal rotation 4D, a vertical rotation 5D and a cyclotorsion 6D can provide an overall six-dimensional orientation of the eye 16 in the space, which can be determined from the eye recordings by the computing device 18.

In order that the computing device 18 can determine these 6 dimensions from the recording data, a structure of the eye, in particular landmarks of the eye, can be predetermined in case of presence of only one camera 26, wherein they can be ascertained in the eye recordings. The landmarks ascertained in the eye recordings, in particular a position and/or a distortion/shearing, can then be compared to the position and/or distortion/shearing of the predetermined landmarks, and it can be examined if and to what extent, respectively, they deviate from each other. By this deviation, the eye orientation can then be ascertained. In particular, a limbus can be measured in the eye recording data, for example a radius or diameter at least in horizontal and vertical direction. Subsequently, it can be compared to the predetermined landmarks of the limbus to determine how far the eye 16 is away from the treatment apparatus and if a rotation in horizontal or vertical direction is present. For example, if the radius is smaller in all spatial directions than in the predetermined landmarks, the eye 16 is farther away from the reference point. If a rotation is present, the radius can change in the corresponding direction, which means that the originally circular limbus is represented as an ellipse or oval in the eye recording data. Based on this deformation, the eye rotation in the corresponding direction can then be inferred.

After the computing device 18 has ascertained the eye orientation in the six dimensions, the computing device 18 can generate control data for controlling the treatment apparatus 10, in which a possible deviation from an optimized eye orientation is compensated for. In particular, the control data can control the beam deflection device 22 such that the laser beam 20 is oriented to originally planned irradiation positions. In particular, this can also be performed during the treatment, in which the eye orientation can be monitored by the capturing device 24 in real time.

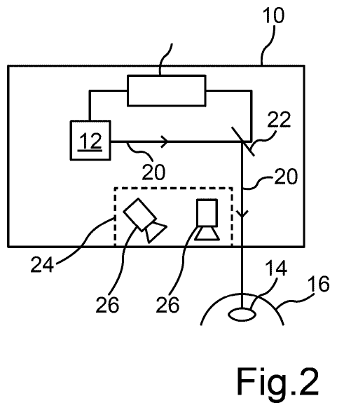

In FIG. 2, a further exemplary embodiment of the treatment apparatus 10 is illustrated. In this embodiment, the capturing device 24 can comprise at least two cameras 26, which capture recordings of the eye 16.

Herein, the cameras 26 of the capturing device 24 can be oriented to the eye 16 at different preset angles to thus record the eye 16 from different positions. In particular, further cameras with further angles can be provided. Due to the different recording angles to the eye 16, the computing device 18 can thus determine depth information, by which the eye orientation in the six dimensions can be ascertained.

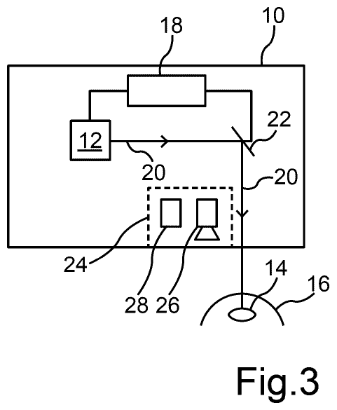

In FIG. 3, a further exemplary embodiment of the treatment apparatus 10 is illustrated. In this embodiment, it can be provided that the capturing device 24 comprises a projection unit 28 for ascertaining the eye orientation besides the camera 26, which can be formed to radiate a light pattern or projection pattern to the eye 16. For example, the projection pattern can be a line or grid pattern with preset distance between the respective lines. From a reflection of this projection pattern, which can be captured by the camera 26, the computing device 18 can determine a distance of the eye 16 as well as vertical or horizontal rotations. For example, a translation and cyclotorsion can then be additionally ascertained based on landmarks in the eye 16, in particular based on a pattern of an iris of the eye 16, and/or a pupil position. In order to ascertain the vertical and horizontal rotation, the distance of the line or stripe pattern can herein be examined, wherein the lines take greater distances in rotational direction upon a rotation of the eye.

In particular, the features of the embodiments, which are described in FIGS. 1 to 3, can also be combined with each other such that an improved determination of the eye orientation is allowed.

Overall, the examples show how an eye orientation in six dimensions can be ascertained by the invention.

1. A treatment apparatus for treating a human and/or animal eye, comprising:
at least one ophthalmological laser for irradiating the eye by laser radiation;
a capturing device with at least one camera, wherein the capturing device is configured to capture eye recording data by the at least one camera; and
a computing device, which is configured to ascertain an eye orientation from the eye recording data, wherein the eye orientation includes a distance, a vertical displacement and a horizontal displacement of the eye in relation to a preset reference point of the at least one ophthalmological laser, a cyclotorsion, a vertical rotation and a horizontal rotation of the eye,
wherein the computing device is further configured to generate control data for controlling the treatment apparatus depending on the ascertained eye orientation.

2. The treatment apparatus according to claim 1, wherein the capturing device includes a projection unit, which is configured to radiate a projection pattern, wherein the computing device is configured to ascertain the eye orientation depending on the projection pattern captured in the eye recording data.

3. The treatment apparatus according to claim 2, wherein the projection pattern is a stripe pattern with preset distances between the stripes.

4. The treatment apparatus according to claim 1, wherein the capturing device comprises at least two cameras, which are arranged to record the eye recording data from different angles.

5. The treatment apparatus according to claim 1, wherein landmarks of the eye are predetermined, wherein the computing device is configured for determining the eye orientation depending on a position of the landmarks in the eye recording data.

6. The treatment apparatus according to claim 1, wherein the computing device is configured to monitor a centering of the eye on a contact element of the treatment apparatus with the control data.

7. The treatment apparatus according to claim 1, wherein the computing device is configured to adapt irradiation positions of a laser beam of the at least one ophthalmological laser with the control data, in particular during a treatment.

8. A method for determining an eye orientation of a human and/or animal eye for a treatment apparatus with an ophthalmological laser, wherein eye recording data of the eye is captured by a capturing device, which includes at least one camera, wherein an eye orientation is ascertained from the eye recording data by a computing device, wherein a distance, a vertical displacement and a horizontal displacement of the eye in relation to a preset reference point of the ophthalmological laser, as a cyclotorsion, a vertical rotation and a horizontal rotation of the eye are determined as the eye orientation, wherein control data for controlling the treatment apparatus is generated depending on the ascertained eye orientation by the computing device.

9. (canceled)

10. A computer-readable medium for storing a computer program, the computer program comprising commands which cause a treatment apparatus to execute the method according to claim 8.
